(19) United States
(12) Patent Application Publication
(10) Pub. No.: US 2017/0216082 A1
(43) Pub. Date: Aug. 3, 2017

(54) Urine Collection Assembly

(71) Applicant: Henry F. Liscio, Jr., Henrico, VA (US)

(72) Inventor: Henry F. Liscio, Jr., Henrico, VA (US)

(21) Appl. No.: 15/008,791

(22) Filed: Jan. 28, 2016

(51) Int. Cl. A61F 5/453; A61F 5/44

(52) U.S. Cl. CPC A61F 5/453; A61F 5/4401; A61F 5/4408

(57) ABSTRACT

A urine collection assembly includes a sock that may be worn on a penis thereby facilitating the sock to collect urine. The sock includes a strap and the strap is matable to itself. The strap is wrapped around the penis thereby removably coupling the sock to the penis. The sock is comprised of a fluid absorbent material to absorb the urine. Thus, the urine is inhibited from penetrating clothing.

### BACKGROUND OF THE DISCLOSURE

### Field of the Disclosure

The disclosure relates to collection devices and more particularly pertains to a new collection device for collecting urine that drips from a penis after urination.

### SUMMARY OF THE DISCLOSURE

An embodiment of the disclosure meets the needs presented above by generally comprising a sock that may be worn on a penis thereby facilitating the sock to collect urine. The sock includes a strap and the strap is matable to itself. The strap is wrapped around the penis thereby removably coupling the sock to the penis. The sock is comprised of a fluid absorbent material to absorb the urine. Thus, the urine is inhibited from penetrating clothing.

There has thus been outlined, rather broadly, the more important features of the disclosure in order that the detailed description thereof that follows may be better understood. and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF THE DRAWINGS

The disclosure will be better understood and objects other than those set forth above will become apparent when consideration is given to the following detailed description thereof. Such description makes reference to the annexed drawings wherein:

FIG. 1 is a top perspective view of a urine collection assembly according to an embodiment of the disclosure.

FIG. 2 is a front view of an embodiment of the disclosure.

FIG. 3 is a bottom view of an embodiment of the disclosure.

FIG. 4 is a cross sectional view taken along line 4-4 of FIG. 3 of an embodiment of the disclosure.

FIG. 5 is a perspective in-use view of an embodiment of the disclosure.

### DESCRIPTION OF THE PREFERRED EMBODIMENT

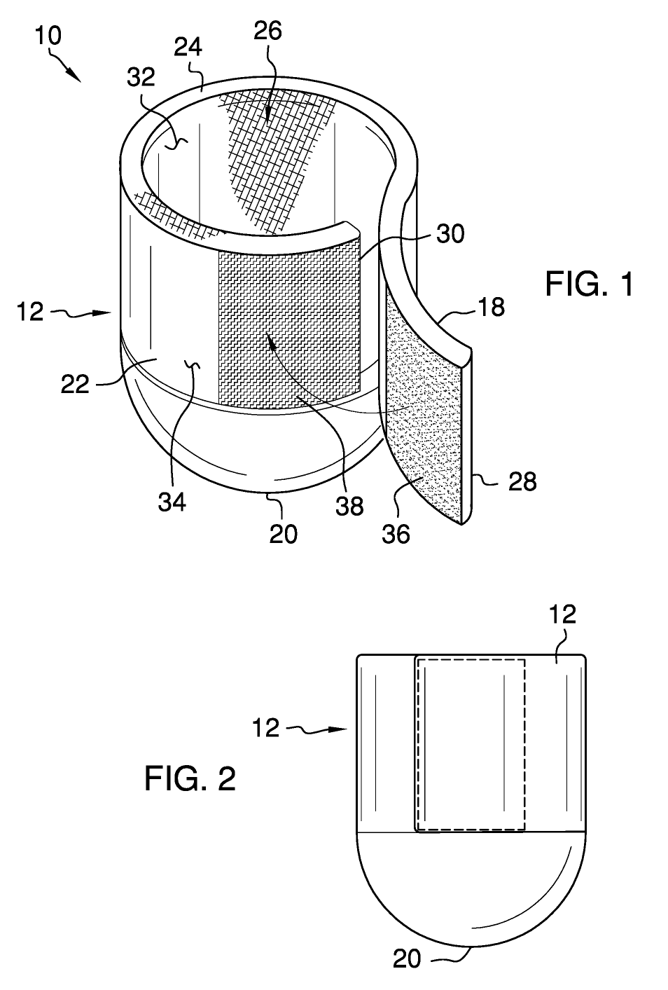

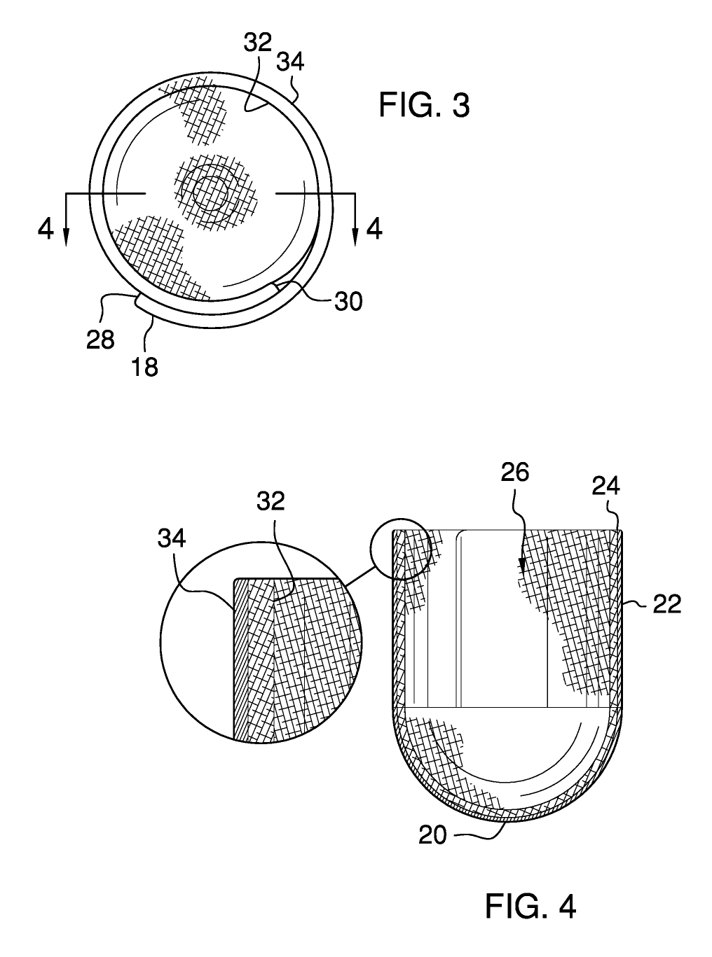

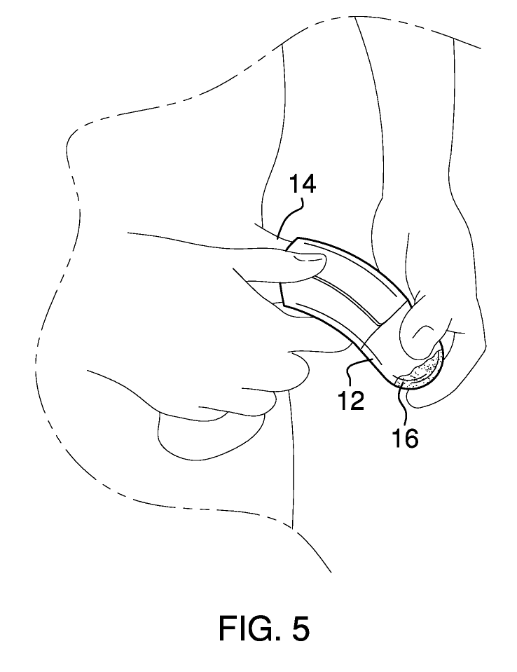

With reference now to the drawings, and in particular to FIGS. 1 through 5 thereof, a new collection device embodying the principles and concepts of an embodiment of the disclosure and generally designated by the reference numeral 10 will be described.

As best illustrated in FIGS. 1 through 5, the urine collection assembly 10 generally comprises a sock 12 that may be worn on a penis 14 thereby facilitating the sock 12 to collect urine 16. The sock 12 includes a strap 18 and the strap 18 is matable to itself. The strap 18 is wrapped around the penis 14 thereby removably coupling the sock 12 to the penis 14. The sock 12 is comprised of a fluid absorbent material such as cotton or the like. Thus, sock 12 absorbs the urine 16 thereby inhibiting the urine 16 from penetrating clothing.

The sock 12 has a bottom end 20 and an outer wall 22 extending away from the bottom end 20. The bottom end 20 is rounded and the outer wall 22 has a distal end 24 with respect to the bottom end 20. The distal end 24 forms an opening 26 in the sock 12 to receive the penis 14. The outer wall 22 is substantially longitudinally split between the distal end 24 and the bottom end 20. Thus each of a first end 28 and a second end 30 of the strap 18 are defined. The first end 28 overlaps the second end 30, and the outer wall 22 has an inner surface 32 and an outer surface 34. The outer surface 34 may be comprised of a deformable material such as paper or the like.

A first coupler 36 is coupled to the strap 18. The first coupler 36 is positioned on the inner surface 32 corresponding to the first end 28. A second coupler 38 is coupled to the strap 18. The second coupler 38 is positioned on the outer surface 34 corresponding to the second end 30. The first coupler 36 is complementary with respect to the second coupler 38. Thus, the strap 18 removably retains the sock 12 on the penis 14.

In use, the sock 12 is worn on the penis 14 after urination. Thus, the sock 12 captures urine 16 that drips from the penis 14 after urination. The penis 14 is inserted into the opening 26 and the penis 14 is extended toward the bottom end 20. The strap 18 is snuggly fitted around the penis 14 and the first coupler 36 is mated to the second coupler 38. Thus, the sock 12 is removably retained on the penis 14. The sock 12 is removed from the penis 14 and discarded when the sock 12 becomes soaked with urine 16.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A urine collection assembly comprising:
a sock being configured to be worn on a penis thereby facilitating said sock to collect urine, said sock including a strap, said strap being matable to itself, said strap being configured to be wrapped around the penis thereby removably coupling said sock to the penis, said sock being comprised of a fluid absorbent material wherein said sock is configured to absorb the urine thereby inhibiting the urine from penetrating clothing.

2. The assembly according to claim 1, wherein said sock has a bottom end and an outer wall extending away from said bottom end, said bottom end being rounded, said outer wall having a distal end with respect to said bottom end, said distal end forming an opening in said sock wherein said opening is configured to receive the penis, said outer wall being substantially longitudinally split between said distal end and said bottom end to define a first end and a second end of said strap, said first end overlapping said second end, said outer wall having an inner surface and an outer surface.

3. The assembly according to claim 2, further comprising a first coupler being coupled to said strap, said first coupler being positioned on said inner surface corresponding to said first end.

4. The assembly according to claim 2, further comprising:
a first coupler; and
a second coupler being coupled to said strap, said second coupler being positioned on said outer surface corresponding to said second end, said first coupler being complementary with respect to said second coupler wherein said strap is configured to removably retain said sock on the penis.

5. A urine collection assembly comprising:
a sock being configured to be worn on a penis thereby facilitating said sock to collect urine, said sock including a strap, said strap being matable to itself, said strap being configured to be wrapped around the penis thereby removably coupling said sock to the penis, said sock being comprised of a fluid absorbent material wherein said sock is configured to absorb the urine thereby inhibiting the urine from penetrating clothing, said sock having a bottom end and an outer wall extending away from said bottom end, said bottom end being rounded, said outer wall having a distal end with respect to said bottom end, said distal end forming an opening in said sock wherein said opening is configured to receive the penis, said outer wall being substantially longitudinally split between said distal end and said bottom end to define a first end and a second end of said strap, said first end overlapping said second end, said outer wall having an inner surface and an outer surface;
a first coupler being coupled to said strap, said first coupler being positioned on said inner surface corresponding to said first end; and
a second coupler being coupled to said strap, said second coupler being positioned on said outer surface corresponding to said second end, said first coupler being complementary with respect to said second coupler wherein said strap is configured to removably retain said sock on the penis.
